(19) United States
(12) Patent Application Publication
(10) Pub. No.: US 2022/0383980 A1
(43) Pub. Date: Dec. 1, 2022

(54) Processing sequencing data relating to amyotrophic lateral sclerosis

(71) Applicant: GenieUs Genomics Pty Ltd, New South Wales (AU)

(72) Inventors: John SU, New South Wales (AU); Matt KEON, New South Wales (AU); Ted WONG, New South Wales (AU); Boris GUENNEWIG, New South Wales (AU)

(21) Appl. No.: 17/825,979

(22) Filed: May 26, 2022

(30) Foreign Application Priority Data
May 26, 2021 (AU) 2021901584

(51) Int. Cl. G16B 30/00; G16B 40/00; G16H 50/30

(52) U.S. Cl. CPC G16B 30/00; G16B 40/00; G16H 50/30

(57) ABSTRACT

This disclosure relates to computationally efficient processing of sequencing data relating to amyotrophic lateral sclerosis (ALS). A processor receives unaligned training reads and determines training sub-sequences from them. The processor then counts the training sub-sequences in a control group and in a group diagnosed with ALS and determines a measure of change, for each of the training sub-sequences, in the counting between the control group and the group with ALS. The processor further selects a subset of training sub-sequences that are distal from a mean value of the measure of change and then receives testing sequencing data comprising multiple unaligned testing reads. The processor determines sub-sequences from the testing reads, counts the sub-sequences that are in the subset, and determines a diagnostic output value related to ALS for the sample based on the counting of the testing sub-sequences that are in the subset.

### TECHNICAL FIELD

This disclosure relates to computationally efficient processing of sequencing data relating to amyotrophic lateral sclerosis.

### BACKGROUND

Amyotrophic lateral sclerosis (ALS) has been the subject of a number of studies and a range of options exist for diagnosis. In some cases, researchers attempt to find genetic markers by whole genome sequencing (WGS) and finding markers in the genome that correlate highly with ALS. However, these methods require alignment of reads from a sequencer to a reference genome so that the genetic marker can be found. The alignment process is computationally very expensive, which means that aligning the reads takes a long time on a high performance computer.

There is a need for a method that reduces this computational time while, at the same time, still providing a reliable prediction of ALS.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

A computer-implemented method for processing sequencing data of multiple subjects comprises:

receiving training sequencing data comprising multiple unaligned training reads from samples of a control group and samples diagnosed with ALS;

determining training sub-sequences from the multiple unaligned training reads;

counting the training sub-sequences in the control group and in the group diagnosed with ALS;

determining a measure of change, for each of the training sub-sequences, in the counting between the control group and the group with ALS;

selecting a subset of training sub-sequences that are distal from a mean value of the measure of change;

receiving testing sequencing data comprising multiple unaligned testing reads from a sample to be tested for ALS;

determining testing sub-sequences from the multiple unaligned testing reads;

counting the testing sub-sequences that are in the subset;

determining a diagnostic output value related to ALS for the sample based on the counting of the testing sub-sequences that are in the subset.

It is an advantage that counting the sub-sequences and using the counts to determine a diagnostic output value is computationally efficient as it bypasses the alignment process. Further, the results are shown to be accurate.

In some embodiments, the training reads have a length of less than 300 bases.

In some embodiments, receiving the training sequences comprises reading a file from computer storage in FASTQ format.

In some embodiments, determining the training sub-sequences comprises selecting a range of base pairs from the training reads.

In some embodiments, the range has a constant length for the training sub-sequences.

In some embodiments, the range is non-overlapping between different sub-sequences.

In some embodiments, counting comprises calculating a counter value for each of the training sub-sequences; and determining a measure of change comprises calculating a difference between the counter value of a sub-sequence in the control group and the counter value of the same sub-sequence in the group diagnosed with ALS.

In some embodiments, the method further comprises normalising the measure of change by adjusting the mean value towards zero.

In some embodiments, adjusting the mean value comprises scaling up one of the control group and the group diagnosed with ALS with a lower abundance in the training sequencing data.

In some embodiments, the method further comprises removing sub-sequences with a low abundance in the training sequencing data.

In some embodiments, selecting the subset comprises selecting training sub-sequences that are more than a threshold distance from the mean value.

In some embodiments, the threshold distance is measured as a log-fold change.

In some embodiments, determining the diagnostic output value comprises: comparing the counting of the testing sub-sequences in the subset to the counting from the control group of the training sub-sequences in the subset and to the counting from the group diagnosed with ALS of the training sub-sequences in the subset.

In some embodiments, the method further comprises upon determining that the counting of the testing sub-sequences in the subset is closer to the counting from the control group of the training sub-sequences in the subset than to the counting from the group diagnosed with ALS of the training sub-sequences in the subset, determining the diagnostic output value that indicates that the sample is diagnosed as not having ALS; and upon determining that the counting of the testing sub-sequences in the subset is closer to the counting from the group diagnosed with ALS of the training sub-sequences in the subset than to the counting from the control group of the training sub-sequences in the subset, determining the diagnostic output value that indicates that the sample is diagnosed as having ALS.

Software, when executed by a computer, causes the computer to perform the method of any one of the preceding claims.

A system for processing sequencing data of multiple subjects comprises a processor configured to perform the steps of:

receiving training sequencing data comprising multiple unaligned training reads from samples of a control group and samples diagnosed with ALS;

determining training sub-sequences from the multiple unaligned training reads;

counting the training sub-sequences in the control group and in the group diagnosed with ALS;

determining a measure of change, for each of the training sub-sequences, in the counting between the control group and the group with ALS;

selecting a subset of training sub-sequences that are distal from a mean value of the measure of change;

receiving testing sequencing data comprising multiple unaligned testing reads from a sample to be tested for ALS;

determining testing sub-sequences from the multiple unaligned testing reads;

counting the testing sub-sequences that are in the subset;

determining a diagnostic output value related to ALS for the sample based on the counting of the testing sub-sequences that are in the subset.

A computer-implemented method for processing sequencing data comprises:

receiving testing sequencing data comprising multiple unaligned testing reads from a sample to be tested for ALS;

determining testing sub-sequences from the multiple unaligned testing reads;

counting the testing sub-sequences that are in a subset of the testing sub-sequences, wherein the subset contains training sub-sequences that are significant in relation to a count of the training sub-sequences in a control group relative to a count of the training sub-sequences a group diagnosed with ALS; and

determining a diagnostic output value related to ALS for the sample based on the counting of the testing sub-sequences that are in the subset.

### BRIEF DESCRIPTION OF DRAWINGS

An example will now be described with reference to the following drawings:

FIG. 1 illustrates a method for processing sequencing data.

FIG. 2 illustrates these counts of training sub-sequences (k-mers).

FIG. 3 illustrates a distribution of the change in count between control and ALS groups.

FIG. 4 illustrates a computer system for processing sequencing data.

### DESCRIPTION OF EMBODIMENTS

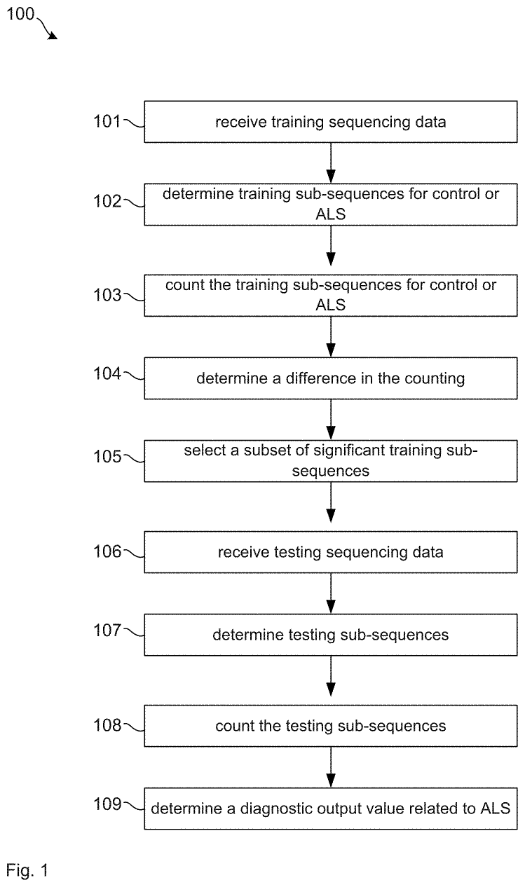

FIG. 1 illustrates a method 100 for processing sequencing data. Method 100 is computer-implemented in the sense that a processor of a computer system performs the method by way of executing program code that implements method 100. Sequencing data relates to data representing a sequence of biological molecules, such as nucleic acids in DNA or RNA.

The processor receives 101 training sequencing data comprising multiple unaligned training reads. The sequencing data may be generated by a whole genome sequencing (WGS) machine, such as an Illumina X10. In this sense, the reads are generated by sequencing by synthesis but other methods are equally possible. Similarly, the reads may be relatively short, such as less than 300 base pairs, 150 bps or longer, such as over 1,000 bps. In one example, the processor receives the sequencing data by reading a file from a file system, such as a FASTQ file.

In one example, the sequencing process is an Illumina dye sequencing, also referred to a sequencing by synthesis, which works in three basic steps: amplify, sequence, and analyse. The process begins with purified DNA. The DNA is fragmented and adapters are added that contain segments that act as reference points during amplification, sequencing, and analysis. The modified DNA is loaded onto a flow cell where amplification and sequencing will take place. The flow cell contains nanowells that space out fragments and help with overcrowding. Each nanowell contains oligonucleotides that provide an anchoring point for the adaptors to attach. Once the fragments have attached, a phase called cluster generation begins. This step makes about a thousand copies of each fragment of DNA and is done by bridge amplification PCR. Next, primers and modified nucleotides are washed onto the chip. These nucleotides have a reversible 3′ fluorescent blocker so the DNA polymerase can only add one nucleotide at a time onto the DNA fragment. After each round of synthesis, a camera takes a picture of the chip. A computer determines what base was added by the wavelength of the fluorescent tag and records it for every spot on the chip. After each round, non-incorporated molecules are washed away. A chemical deblocking step is then used to remove the 3′ fluorescent terminal blocking group. The process is repeated until the full DNA molecule is sequenced. With this technology, thousands of places throughout the genome are sequenced at once via massive parallel sequencing. The result is written into a file, such as in the FASTQ format (see http://maq.sourceforge.net/fastq.shtml).

The FASTQ format is a text-based format for storing both a biological sequence (usually nucleotide sequence) and its corresponding quality scores. Both the sequence letter and quality score are each encoded with a single ASCII character for brevity. However, in other examples, the sequencing data is in other formats, such as a binary format or in the form of a database, such as a relational or SQL database, for example. The entire sequencing data may be received at once as a file, or over time while the analysis is performed. For example, the sequencing data may be received as a real-time stream of sequencing data, such as from a nanopore sequencer, such as sequences provided by Oxford Nanopore Technologies.

The sequencing data comprises reads from multiple samples. These samples are divided into two groups. The first group is a control group with individuals that have not been diagnosed with ALS while the second group has individuals that have been diagnosed with ALS (“ALS group” in short). The groups may be defined in the sequencing data by a flag for each read and the flag is set or unset to indicate that this read is in the control group or the ALS group. There may also be a single flag for the entire set of reads from one sample.

Many bioinformatics pipelines include an alignment step which finds, for each read, one or more positions in a reference genome at which that read has a high similarity with the reference sequence. Ideally, alignment algorithms map each read to exactly one position in the reference genome. This way, differences between the read and the reference genome can be identified as a variant by a variant caller in a subsequent step. However, it is computationally difficult to align relatively short reads of about 150 bps to a reference genome of millions of bps reliably. As a result, many reads are mapped to an incorrect position and the computational complexity is high. Kyu-Baek Hwang, et al.: Comparative analysis of whole-genome sequencing pipelines to minimize false negative findings, Nature Scientific Reports (2019) 9:3219|https://doi.org/10.1038/s41598-019-39108-2 provides a review of 7 short-read aligners and 10 variant calling algorithms and observes “remarkable differences in the number of variants called by different pipelines”. This highlights, that even despite the invested computational effort, the results are inaccurate.

This problem is exacerbated when genome sequencing is used for a large number of samples to train a model. In that scenario, each of potentially thousands of genome sequences need to be aligned and each of those would have hundreds of millions of short reads to align (about 600 million for 30× coverage) against a reference genome.

In order to address this difficulty, in the methods disclosed herein, the training reads are unaligned, which means that the sequence is independent from a reference sequence and it is not known where in the reference sequence each read of the training sequence is located. In other words, there is no association between each of the training reads and a location on a reference sequence or the human genome in general. Put yet another way, the training reads can be read directly from the FASTQ file from the sequencer without any further processing. Therefore, an unaligned training read can be from anywhere in the genome. The reason for this lack of association is that the sequencing process splits the entire genome of one sample into fragments before the sequencing each of the fragments to generate a read. As a result, all fragments in the sample are present together in a mixture without a particular order or association to each other. That is, the connection between the fragments that exists in the intact genome, is lost in the process of splitting the fragments and mixing them.

Next, the processor determines 102 training sub-sequences from the multiple unaligned training reads. Again, of these sub-sequences are either in the control group or the ALS group. The sub-sequences can also be referred to as k-mers, which a sub-sequences of length k. The processor may determine the sub-sequences by further splitting the reads, noting that this is now performed in-silico, that is, on digital data and not directly on chemical molecules. In one example, the processor splits each read into k-mers of equal lengths, starting from the beginning of the read. The length may be greater than 10, greater than 30, exactly 31, between 20 and 50 or between 10 and 100. The training sub-sequences may overlap on the read, so that the first sub-sequence contains bases from position 0-30 of the read, the second sub-sequence from 1-31, the third sub-sequences from 2-32 and so on. In that case, there would be about 4 k-mers per read, which means about 3,200 million k-mers for 30× coverage. These k-mers are counted, which is significantly faster than aligning 600 k-mers.

In another example, the are contiguous and not overlapping. In that case, the first sub-sequences contains bases 0-30 of the read, the second sub-sequence contains bases 31-61, the third sub-sequence contains bases 62-92 and so on. In that case, there would be about 120 k-mers from each read. So for 600 million reads, there would be 72,000 million k-mers. Again, counting this number of k-mers is still significantly faster than aligning 600 millions reads against a reference genome.

In other examples, the processor generates every possible sub-sequence from each read. This means, the processor generates every possible sub-sequence of length k=1, then of length k=2, then of length k=3, and so on. For each length k, there are L-k+1 k-mers (where L is the length of the read).

The processor then counts the training sub-sequences, that is k-mers, in the control group and in the group diagnosed with ALS. In one example, the processor executes the software tool “Jellyfish” (https://github.com/gmarcais/Jellyfish/), which is a tool for fast, memory-efficient counting of k-mers in DNA. Jellyfish can count k-mers using an order of magnitude less memory and an order of magnitude faster than other k-mer counting packages by using an efficient encoding of a hash table and by exploiting the “compare-and-swap” CPU instruction to increase parallelism. In another example, the processor executes DSK (disk streaming of k-mers) available at https://github.com/GATB/dsk. DSK only requires a fixed user-defined amount of memory and disk space. This approach realizes a memory, time and disk trade-off. The multi-set of all k-mers present in the reads is partitioned, and partitions are saved to disk. Then, each partition is separately loaded in memory in a temporary hash table. The k-mer counts are returned by traversing each hash table. Low-abundance k-mers are optionally filtered. DSK is the first approach that is able to count all the 27-mers of a human genome dataset using only 4.0 GB of memory and moderate disk space (160 GB), in 17.9 h. DSK can replace other k-mer counting software (Jellyfish) on small-memory servers. In one example, the processor filters (i.e. removes) the low-abundance k-mers, such as by setting a minimum abundance of 100.

The result of k-mer counting is a list of k-mers and for each k-mer there is a number that indicates the number of times that k-mer has been encountered in the sequencing data. More particularly, this list of k-mers can be produced for each sample, so for 1,000 samples in the control group, there are 1,000 lists of k-mers. These lists can be merged in a variety of different ways. For example, the processor can calculate the mean count for every k-mer, so add the counts from each sample and divide by the number of samples (1,000) in this example. This results in an average count for each sample for the control group. The processor repeats this process of calculating the average count for the ALS group. As a result, there are now two lists of k-mers including one list for the control group and one list for the ALS group. It is noted that whenever is made reference to ‘list’ this could be a different, more efficient data structure, such as a hash table.

The processor then determines a difference, for each of the training sub-sequences, in the count between the control group and the group with ALS. In other words, the processor determines how much more abundant each k-mer is in the two groups. In effect, for k-mer i this is a calculation of dᵢ=c_{i,C}−c_{i,A} where c_{i,C} is the count of k-mer i in control group C and c_{i,A} is the count of k-mer i in ALS group A.

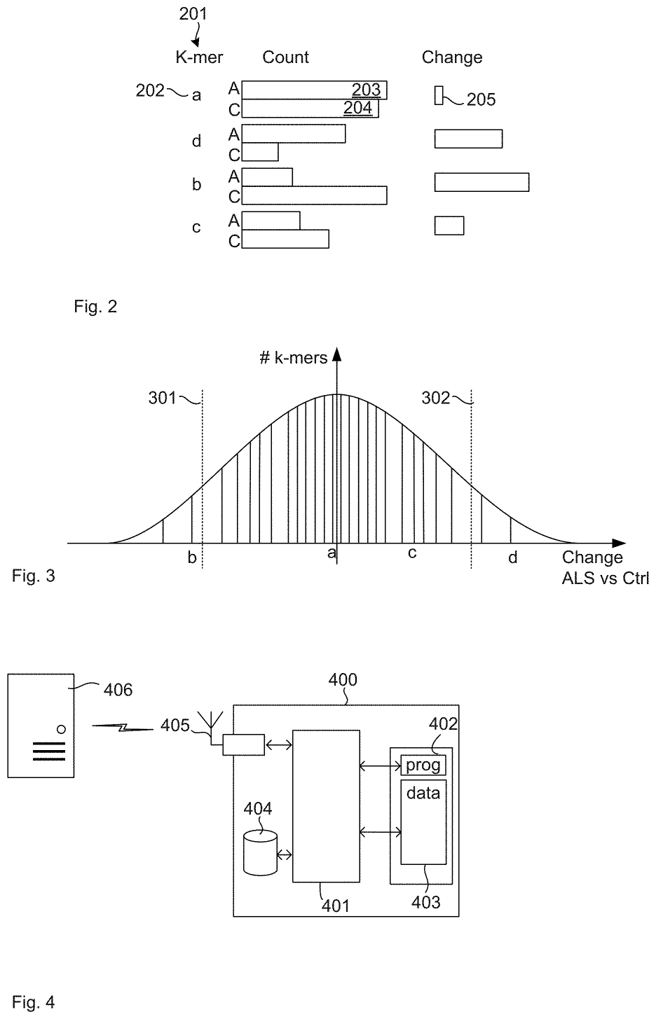

FIG. 2 illustrates these counts. That is, there is a list of k-mers 201, where each k-mer is referenced by a lower case number. So k-mer ‘a’ may be a sub-sequence of “GGTTA”, for example. The count values are indicated by the length of respective bars. So an example first k-mer 202 has a first count 203 for ALS group A and second count 204 for control group C. Similarly, the remaining k-mers b, b and c have corresponding count values. FIG. 2 also shows the change 205 in count values, which geometrically is the difference in length between the bar for the first count 203 and the second count 204. The algebraic difference is used here for an illustrative example, but in other examples, the processor calculates a ratio or a log-fold change, that is

$$log ( \frac{{c}_{i , A}}{{c}_{i , C}} ) .$$

It should be noted that where both counts are identical, the ratio is 1 and the log of 1 is zero. So if both counts are different, the log-fold change is 0, which is intuitive. In some example, base 2 is used for the logarithm and the result is then referred to a log 2-fold change or simply log-fold change.

If a k-mer is not present in one of the samples, because it has been filtered, for example, it is deleted from the entire count data. From FIG. 2, it can be seen that k-mers ‘a’ and ‘c’ have a relatively small change between the control group and the ALS group while k-mers ‘d’ and ‘b’ have a relatively large change.

The values for change in count value can be represented in a distribution, which is shown in FIG. 3, where the x-axis is the log-fold change. More specifically, each k-mer is represented by a vertical line, so k-mer ‘a’ from FIG. 2 is located relatively close to the centre while k-mers ‘b’ and ‘d’ are located relatively distal from the centre. In FIG. 3, the k-mers have been binned along the x-axis to generate a histogram indicated by the solid bell-curve. This indicates that there is a large number of k-mers near the centre but only a small number of k-mers on the outside (tail) of the bell-curve. FIG. 3 is an illustration only and processor does not necessarily need to construct the actual distribution as shown in FIG. 3.

It is useful, however, for the processor to calculate the mean value of all change values, noting that they can be positive or negative. Generally, the mean value should be zero because most k-mers should have an identical count between the control group and the ALS group. The reason for this is that the genome across any subjects are identical except a small number of regions that differ. If the mean calculated by the processor is not zero, the processor can normalise the distribution. This normalisation can be achieved by scaling up the group with a lower abundance. That is, processor multiplies the counts of one group by a factor that is constant for all k-mers of that group. In some experiments, the group for scaling up was the group with the lower abundance across all k-mers, which was the control group in some cases.

In one example, the processor does not calculate an average of counts as stated above. Instead, the processor simply adds all the counts from all samples into one large count for each k-mer. The processor does this for each of the control group and the ALS group to create two count values for each k-mer. The processor then calculates the measure of change by calculating the logarithm of the ratio of the count from the ALS group over the count value of the control group, noting that these count values have not been divided by the number of samples. So if the control group is larger, the count value of the control group would be naturally larger on ever k-mer. However, scaling up the count values from the group with the smaller abundance (smaller number of samples) automatically corrects for this asymmetry in group sizes. This scaling can be continued until the mean value of the count differences is zero. The processor can determine the optimal scaling factor by a gradient descent method, a binary search or any other optimisation method.

Once the count values (potentially normalised) are available for each k-mer, the processor can select 105 a subset of significant k-mers that are distal from a mean value of the difference for the training sub-sequences. For the case of a normalised distribution, the mean value is zero, so the processor selects k-mers that are distal from zero, which could be negative or positive. The definition of distal can present a trade-off between significance of the k-mers against number of selected k-mers. In one example, the difference between counts is expressed in a log-fold change of at least +/−2, noting that low-abundance k-mers have been filtered. In the example of FIG. 3, this log-fold threshold has been indicated by dotted lines 301, 302.

As a result, the processor selects k-mers ‘b’ and ‘d’ in the example of FIG. 3. It is noted here again, that the selected k-mers could be of any length and from anywhere in the genome. It is even possible that the k-mer count includes k-mers that are identical but from completely different genomes as they can be from different reads.

With the identified significant k-mers (those with a relatively large change value), the processor can now receive testing sequencing data comprising multiple unaligned testing reads from a sample to be tested for ALS. This sequencing data can be formally identical to the training sequencing data that has been used to select the significant k-mers. So the testing sequencing data may also be in the form of a FASTQ file. However, the testing sequencing data is only from a single individual.

Again, the processor determines testing sub-sequences from the multiple unaligned testing reads as described above, which may involve generating all k-mers from the testing reads or fixed-length k-mers. The processor also counts the testing sub-sequences that are in the subset. In the above example, this means that processor counts the k-mers ‘b’ and ‘d’.

Finally, the processor determines a diagnostic output value related to ALS for the sample based the counting of the testing sub-sequences that are in the subset. For example, the processor determines whether the count value of these k-mers is closer to the average count value in the control group than to the average count value in the ALS group. If that is the case, the processor determines a diagnostic output value that indicates that this individual is unlikely to have ALS. Conversely, if the count value of these k-mers is closer to the average count value in the ALS group than to the average count value in the control group, the processor indicates that the individual likely has ALS.

### Experiments

The method has been tested on sequencing data of the AnswerALS genomic samples from https://dataportal.answerals.org/. All samples where downloaded in FASTQ format together with their labels and the entire AnswerALS dataset has been used.

We applied the filtering analysis described earlier, and constructed a residual neural network (RNN) with gated recurrent units (GRU) to predict the significant k-mers between ALS and controls. The model inputs are one-hot encoded with fixed length of 31. The network provides a binary classification with a binary cross entropy loss function and an Adams optimizer. We trained the RNN on all of the k-mers from the entire dataset, which comprises sampled labelled as ALS and control to train the RNN as a binary classifier. Then, we used the k-mers that were selected as significant by the methods disclosed herein as input to be classified to the RNN. For most of the selected k-mers, the RNN classified them as ALS significant, which indicates that the disclosed method indeed selects k-mers that are correlated to the biological observations. More specifically, we achieved an F1 score of about 94%, and hence sufficient statistical evidence to show the k-mers we derived are biologically correlated.

The RNN method was used since the disclosed methods are able to select significant k-mers but the selected k-mers may not have anything common in it, they could just be random. In order to show that they are not random but inherit a relationship, we use the GRU RNN machine learning to show the k-mers are indeed correlated.

Provided below is a simple example with two set of k-mers:

1. “ABCDEF”, “SDHSDJ”

2. “ABCDEF”, “BCDEFG”

The first set of k-mers are random. There doesn't seem to be any relationship between “ABCDEF” to “SDHSDJ”. However, the relationship is clear in the second case. We use the machine learning model to show the k-mers we have are linked like in the second case (not the first case).

We repeated the same methods on New York Genome, the entire dataset. That is, we applied the same method as for the AnswerALS dataset on this different dataset. We have shown the GRU RNN model was able to score >90% F1 score in the binary classification between ALS and controls, implying the k-mers we generated are correlated. If there was no correlation (k-mers could be just random), the performance of the model should have hovered around 50%.

### Implementation

FIG. 4 illustrates a computer system 400 for processing sequencing data. The computer system 400 comprises a processor 401 connected to a program memory 402, a data memory 403, a database 404 and a communication port 405. The program memory 402 is a non-transitory computer readable medium, such as a hard drive, a solid state disk or CD-ROM. Software, that is, an executable program stored on program memory 402 causes the processor 401 to perform the method in FIG. 1, that is, processor 401 receives sequencing data, determines a measure of change in counts between the control and ALS group to then select significant k-mers. Processor 401 then uses the selected k-mers to diagnose a test sample. The term “determining a measure” refers to calculating a value that is indicative of the measure. This also applies to related terms. In one example, the program is a C++ program based on the open source Kallisto software that computes the k-mer counts efficiently.

The processor 401 may then store the selected k-mers and other generated data, including a determined diagnostic output value, on data store 403, such as on RAM or a processor register, or on database 404. Processor 402 may also send the determined data or the diagnostic output value via communication port 405 to a server, such as a patient data server or electronic health record server.

The processor 401 may receive data, such as the sequencing data, from data memory 403 as well as from the communications port 405, which is connected to a sequencer 406, such as an Illumina X10 sequencer. In another example, there is a shared storage available, such as cloud storage, on which sequencer 406 writes the sequencing data, such as by creating a FASTQ file. Processor 401 then receives the sequencing data by reading the file from cloud storage.

In one example, the processor 401 receives and processes the sequencing data in real time. This means that the processor 401 determines the count for the test sequence every time a new base pair is received from the sequencer and completes this calculation before the sequencer sends the next sequencing update. In another example, the processor 401 processes the sequencing data each time sufficient sequences are available for another sub-sequence (k-mer). So if the k-mer length is fixed at 31 bps, processor 401 can increment a counter for the received k-mer as soon as all 31 bps have been received, instead of waiting for the full 150 bps read to arrive. This way, sequencing can be stopped as soon as a diagnostic value has been determined.

Although communications port 405 is shown as a distinct component, it is to be understood that any kind of data port may be used to receive data, such as a network connection, a memory interface, a pin of the chip package of processor 401, or logical ports, such as IP sockets or parameters of functions stored on program memory 402 and executed by processor 401. These parameters may be stored on data memory 403 and may be handled by-value or by-reference, that is, as a pointer, in the source code.

The processor 401 may receive data through all these interfaces, which includes memory access of volatile memory, such as cache or RAM, or non-volatile memory, such as an optical disk drive, hard disk drive, storage server or cloud storage. The computer system 400 may further be implemented within a cloud computing environment, such as a managed group of interconnected servers hosting a dynamic number of virtual machines.

It is to be understood that any receiving step may be preceded by the processor 401 determining or computing the data that is later received. For example, the processor 401 determines a sequencing data and stores the sequencing data in data memory 403, such as RAM or a processor register, or database 404. The processor 401 then requests the data from the data memory 403 or database 404, such as by providing a read signal together with a memory address. The data memory 403 provides the data as a voltage signal on a physical bit line and the processor 401 receives the data via a memory interface.

It is to be understood that throughout this disclosure unless stated otherwise, values, sets, sequences, and the like refer to data structures, which are physically stored on data memory 403 or processed by processor 401. Further, for the sake of brevity when reference is made to particular variable names, such as “measure of change”, this is to be understood to refer to values of variables stored as physical data in computer system 400.

FIG. 1 is to be understood as a blueprint for the software program and may be implemented step-by-step, such that each step in FIG. 1 is represented by a function in a programming language, such as C++ or Java. The resulting source code is then compiled and stored as computer executable instructions on program memory 402.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A computer-implemented method for processing sequencing data of multiple subjects, the method comprising:
receiving training sequencing data comprising multiple unaligned training reads from samples of a control group and samples diagnosed with ALS;
determining training sub-sequences from the multiple unaligned training reads;
counting the training sub-sequences in the control group and in the group diagnosed with ALS;
determining a measure of change, for each of the training sub-sequences, in the counting between the control group and the group with ALS;
selecting a subset of training sub-sequences that are distal from a mean value of the measure of change;
receiving testing sequencing data comprising multiple unaligned testing reads from a sample to be tested for ALS;
determining testing sub-sequences from the multiple unaligned testing reads;
counting the testing sub-sequences that are in the subset;
determining a diagnostic output value related to ALS for the sample based on the counting of the testing sub-sequences that are in the subset.

2. The method of claim 1, wherein the training reads have a length of less than 300 bases.

3. The method of claim 1, wherein receiving the training sequences comprises reading a file from computer storage in FASTQ format.

4. The method of claim 1, wherein determining the training sub-sequences comprises selecting a range of base pairs from the training reads.

5. The method of claim 4, wherein the range has a constant length for the training sub-sequences.

6. The method of claim 4, wherein the range is non-overlapping between different sub-sequences.

7. The method of claim 1, wherein
counting comprises calculating a counter value for each of the training sub-sequences; and
determining a measure of change comprises calculating a difference between the counter value of a sub-sequence in the control group and the counter value of the same sub-sequence in the group diagnosed with ALS.

8. The method of claim 1, wherein the method further comprises normalising the measure of change by adjusting the mean value towards zero.

9. The method of claim 8, wherein adjusting the mean value comprises scaling up one of the control group and the group diagnosed with ALS with a lower abundance in the training sequencing data.

10. The method of claim 1, wherein the method further comprises removing sub-sequences with a low abundance in the training sequencing data.

11. The method of claim 1, wherein selecting the subset comprises selecting training sub-sequences that are more than a threshold distance from the mean value.

12. The method of claim 11, wherein the threshold distance is measured as a log-fold change.

13. The method of claim 1, wherein determining the diagnostic output value comprises comparing the counting of the testing sub-sequences in the subset to the counting from the control group of the training sub-sequences in the subset and to the counting from the group diagnosed with ALS of the training sub-sequences in the subset.

14. The method of claim 13, wherein the method further comprises:
upon determining that the counting of the testing sub-sequences in the subset is closer to the counting from the control group of the training sub-sequences in the subset than to the counting from the group diagnosed with ALS of the training sub-sequences in the subset, determining the diagnostic output value that indicates that the sample is diagnosed as not having ALS; and
upon determining that the counting of the testing sub-sequences in the subset is closer to the counting from the group diagnosed with ALS of the training sub-sequences in the subset than to the counting from the control group of the training sub-sequences in the subset, determining the diagnostic output value that indicates that the sample is diagnosed as having ALS.

15. A non-transitory computer-readable medium with program code stored thereon that, when executed by a computer, causes the computer to perform the method of claim 1.

16. A system for processing sequencing data of multiple subjects, the system comprising a processor configured to perform the steps of:
receiving training sequencing data comprising multiple unaligned training reads from samples of a control group and samples diagnosed with ALS;
determining training sub-sequences from the multiple unaligned training reads;
counting the training sub-sequences in the control group and in the group diagnosed with ALS;
determining a measure of change, for each of the training sub-sequences, in the counting between the control group and the group with ALS;
selecting a subset of training sub-sequences that are distal from a mean value of the measure of change;
receiving testing sequencing data comprising multiple unaligned testing reads from a sample to be tested for ALS;
determining testing sub-sequences from the multiple unaligned testing reads;
counting the testing sub-sequences that are in the subset;
determining a diagnostic output value related to ALS for the sample based on the counting of the testing sub-sequences that are in the subset.

17. A computer-implemented method for processing sequencing data, the method comprising:
receiving testing sequencing data comprising multiple unaligned testing reads from a sample to be tested for ALS;
determining testing sub-sequences from the multiple unaligned testing reads;
counting the testing sub-sequences that are in a subset of the testing sub-sequences, wherein the subset contains training sub-sequences that are significant in relation to a count of the training sub-sequences in a control group relative to a count of the training sub-sequences a group diagnosed with ALS; and
determining a diagnostic output value related to ALS for the sample based on the counting of the testing sub-sequences that are in the subset.
